(19) United States
(12) Patent Application Publication
(10) Pub. No.: US 2022/0250279 A1
(43) Pub. Date: Aug. 11, 2022

(54) METHOD AND SYSTEM FOR DETECTING SEGREGATION OCCURING IN A FRESH CONCRETE MIXTURE AGITATED IN A MIXER DRUM

(71) Applicant: Command Alkon Incorporated, Birmingham, AL (US)

(72) Inventor: Denis Beaupre, Sainte-Catherine-de-la-Jacques (CA)

(21) Appl. No.: 17/668,967

(22) Filed: Feb. 10, 2022

Related U.S. Application Data
(60) Provisional application No. 63/148,215, filed on Feb. 11, 2021.

(51) Int. Cl. B28C 5/42; B28C 7/02

(52) U.S. Cl. CPC B28C 5/422; B28C 5/4272; B28C 7/02

(57) ABSTRACT

A method for detecting segregation occurring in fresh concrete mixture being agitated in a drum generally includes: rotating said drum about its rotation axis at a low rotational speed for agitating said fresh concrete mixture during at least a rotation; said fresh concrete mixture segregating, said segregating including gravity pulling denser concrete ingredients downwards in said fresh concrete mixture; measuring a plurality of pressure values indicative of pressure exerted onto a rheological probe mounted inside said drum and moving through said fresh concrete mixture as said drum rotates; providing reference data indicative of a behaviour of said rheological probe in a fresh concrete mixture in said absence of said segregating; and detecting that said segregating has occurred, including comparing at least some of said measured pressure values to said reference data.

### CROSS-REFERENCE TO RELATED APPLICATIONS

This application claims the benefit of U.S. provisional application No. 63/148,215, filed 11 Feb. 2021, which is hereby incorporated by reference as though fully set forth herein.

### FIELD

The improvements generally relate to handling fresh concrete mixture inside a rotating drum of a mixer truck, and more particularly relate to detecting segregation as fresh concrete mixture is rotated at a low rotational speed.

### BACKGROUND

Fresh concrete mixture is formed of a mixture of ingredients including at least cement-based material, aggregates and water in given proportions. The ingredients are typically transported inside a drum of a mixer truck where the fresh concrete mixture ingredients can be mixed by rotating the drum at a high rotational speed for a given number of drum rotations. After the mixing phase, the fresh concrete mixture is agitated by rotating the drum at a low rotational speed until it is poured into formwork at a job site. When the fresh concrete mixture achieves an homogeneous suspension during the mixing phase, it is generally assumed to be maintained throughout the subsequent agitation phase. In some cases, an homogeneous suspension can even be completed during the agitation phase if not already achieved by then.

However, for some fresh concrete mixtures, segregation may happen during the agitation phase as gravity gradually pulls the denser concrete ingredients such as the aggregates downwards in the fresh concrete mixture, and reciprocally pushes the liquids and/or cement-based material upwards. For instance, segregation may be more likely when the fresh concrete mixture is not properly proportioned, e.g., too much water, not enough fine particles, and the like. Segregation may also occur if the fresh concrete mixture includes inappropriate aggregates such as high density aggregates, inadequately graded aggregates, etc. If the fresh concrete mixture is subjected to too much vibrations during the agitation phase, or if it is modified with additional water and/or admixtures after the initial ingredient mixing, segregation may also follow.

Segregation is generally detected by visually examining the fresh concrete mixture during slump or slump flow measurements, or by noticing inconsistencies in the aspect of the fresh concrete mixture as it is pumped out of the drum, during the pouring of the fresh concrete mixture into formworks, or at the timely removal of the formworks which may reveal honeycombing or other types of visually identifiable defects. Although existing segregation detection techniques are satisfactory to a certain degree, there remains room for improvement, as the qualities of the hardened concrete originating from a segregated fresh concrete mixture are likely to be far inferior to expectations.

### SUMMARY

It was found that there is a need for methods and systems for detecting segregation in fresh concrete mixture as it is agitated in a drum prior to its pouring into place at a job site.

In accordance with a first aspect of the present disclosure, there is provided a method for detecting segregation occurring in a fresh concrete mixture being agitated in a drum, said drum having a rotation axis, said fresh concrete mixture having denser concrete ingredients, said method comprising: rotating said drum about said rotation axis at a low rotational speed for agitating said fresh concrete mixture during at least a rotation; said fresh concrete mixture segregating, said segregating including gravity pulling said denser concrete ingredients downwards in said fresh concrete mixture; measuring a plurality of pressure values indicative of pressure exerted onto a rheological probe mounted inside said drum and moving through said fresh concrete mixture as said drum rotates; providing reference data indicative of a behaviour of said rheological probe in a fresh concrete mixture in said absence of said segregating; and detecting that said segregating has occurred, including comparing at least some of said measured pressure values to said reference data.

In accordance with a second aspect of the present disclosure, there is provided a system for detecting segregation occurring in fresh concrete mixture being agitated in a drum, said drum having a rotation axis, said fresh concrete mixture having denser concrete ingredients, said system comprising: a driving device driving rotation of said drum about said rotation axis at a low rotational speed for agitating said fresh concrete mixture during at least a rotation, said fresh concrete mixture segregating including gravity pulling said denser concrete ingredients downwards in said fresh concrete mixture; a rheological probe mounted inside said drum and moving through said fresh concrete mixture as said drum rotates, and measuring a plurality of pressure values indicative of pressure exerted onto said rheological probe during said rotation; and a controller being communicatively coupled to said rheological probe, said controller having a processor and a memory having stored thereon instructions that when executed by said processor perform the steps of: accessing reference data indicative of a behaviour of said rheological probe in a fresh concrete mixture in said absence of said segregating; and detecting that said segregating has occurred, including comparing at least some of said measured pressure values to said reference data.

Many further features and combinations thereof concerning the present improvements will appear to those skilled in the art following a reading of the instant disclosure.

### DESCRIPTION OF THE FIGURES

In the figures,

FIG. 1 is a side and sectional view of an example of a system for detecting segregation in a fresh concrete mixture being agitated in a rotating drum, showing a driving device for driving rotation of the drum, a rheological probe mounted inside the rotating drum, and a controller, in accordance with one or more embodiments;

FIG. 2A is a sectional view taken along line 2-2 of FIG. 1, showing the rotating drum when a concrete mixture of a non-segregated state is agitated inside the drum, in accordance with one or more embodiments;

FIG. 2B is a sectional view taken along line 2-2 of FIG. 1, showing the rotating drum when a fresh concrete mixture of a segregated state is agitated inside the drum, in accordance with one or more embodiments;

FIG. 3 is a graph showing rotational speed of the drum of FIG. 2 as a function of time, in accordance with one or more embodiments;

FIG. 3A is a graph pressure values measured by the rheological probe as it rotates through the fresh concrete mixture of FIG. 2A, in accordance with one or more embodiments;

FIG. 3B is a graph pressure values measured by the rheological probe as it rotates through the fresh concrete mixture of FIG. 2B, in accordance with one or more embodiments;

FIG. 4 is a graph showing overlapped pressure value patterns for fresh concrete mixtures of non-segregated and segregated states, in accordance with one or more embodiments;

FIG. 5 is a schematic view of an example of a computing device of the controller of FIG. 1, in accordance with one or more embodiments; and

FIG. 6 is a flow chart of an example of a method for detecting segregation occurring in a fresh concrete mixture being agitated in a rotating drum, in accordance with one or more embodiments.

### DETAILED DESCRIPTION

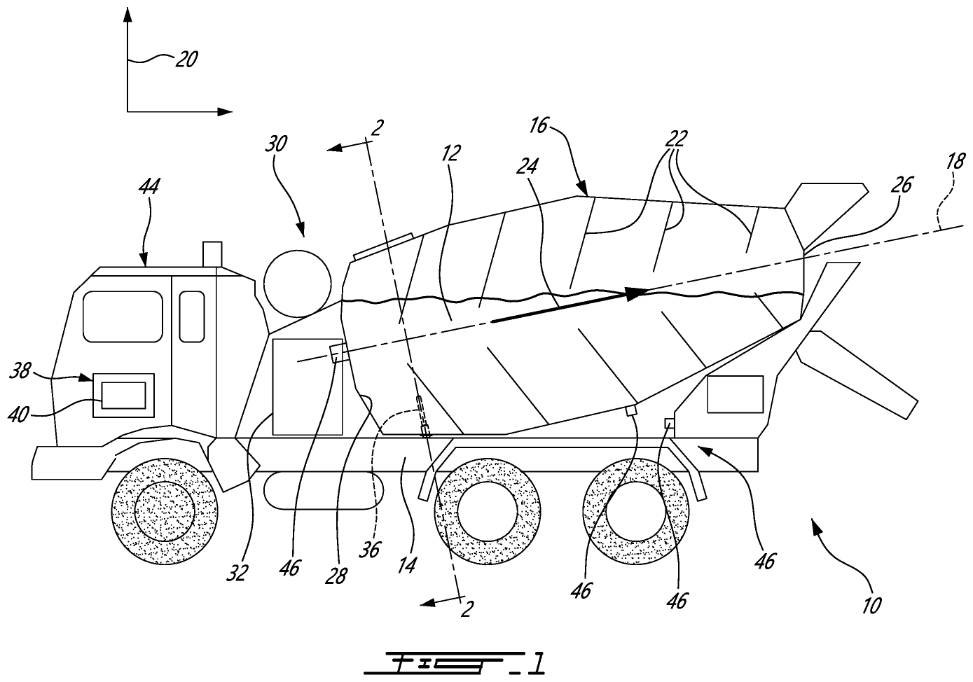

FIG. 1 shows an example of a fresh concrete mixer truck 10 (hereinafter referred to as “mixer truck 10”) for handling fresh concrete mixture 12. As shown, the mixer truck 10 has a frame 14 and a rotating drum 16 which is rotatably mounted to the frame 14. As such, the drum 16 can be rotated about a rotation axis 18 which is at least partially horizontally-oriented relative to the vertical 20.

As illustrated, the drum 16 has inwardly protruding blades 22 mounted inside the drum 16 which, when the drum 16 is rotated in an unloading direction, force the fresh concrete mixture 12 along a discharge direction 24 towards a discharge outlet 26 of the drum 16 so as to be discharged at a job site. In contrast, when the drum 16 is rotated in a mixing direction, opposite to the unloading direction, the fresh concrete mixture 12 is kept and mixed inside the drum 16.

In some embodiments, concrete ingredients (e.g., cement, aggregates and water) are loaded in the drum 16 after which the drum 16 can be rotated a certain number of rotations in the mixing direction at a high rotational speed so as to suitably mix the concrete ingredients to one another, thus yielding the fresh concrete mixture 12. In other embodiments, already mixed fresh concrete mixture is loaded inside the drum 16, in which case the fresh concrete mixture 12 can still be further mixed inside the drum 16 before discharge. Once the concrete ingredients are deemed to be properly mixed, the rotational speed of the drum is generally reduced to a low rotational speed thereby agitating, rather than mixing, the fresh concrete mixture 12 to prevent it to harden prior to arriving at the job site.

In this example, the fresh concrete mixer truck 10 has a system 30 for detecting segregation in the fresh concrete mixture 12 being agitated in the drum 16. As shown, the system 30 has a driving device 32, a rheological probe 36 and a controller 38.

The driving device 32 is mounted to the frame 14 for driving rotation of the drum 16. In this example, the driving device 32 is hydraulic and thus the rotation of the drum 16 is driven using a hydraulic fluid. The driving device 32 can be electrically powered, or powered in any other suitable manner, in some embodiments. The hydraulic fluid can be oil (e.g., mineral oil), water and the like. The driving device 32 exerts a torque on the drum 16, about the rotation axis 18 so as to rotate the drum 16 in any of the unloading and mixing directions. The torque exerted on the drum 16 by the driving device 32 can increase or decrease over time to accelerate or decelerate the rotation of the drum 16, as desired. Typically, the driving device 32 drives the rotation of the drum 16 at a high rotational speed during the mixing phase after which the rotational speed is reduced to a low rotational speed during the agitation phase.

As depicted in this example, the rheological probe 36 is mounted inside the drum 16 and extends in a radial orientation of the drum 16. The rheological probe 36 is configured to measure pressure values as the probe 22 is moved circumferentially through the fresh concrete mixture by the rotation of the drum 16 about the rotation axis 18. As the rheological probe 36 is so moved, it reaches a plurality of circumferential positions at different moments in time, which can be associated with corresponding ones of the pressure values measured by the rheological probe 36. A potential example of the rheological probe 36 is described in international patent publication no. WO 2011/042880, the contents of which are hereby incorporated by reference. However, any other suitable rheological probes, devices or combination of devices that can measure pressure exerted thereon by fresh concrete can be used.

The controller 38 has a processor and a non-transitory memory having instructions stored thereon that when executed by the processor can initiate a sequence for the detection and monitoring of segregation occurring in the fresh concrete mixture 12 being agitated in the drum 16. As such, the controller 38 is communicatively coupled with the rheological probe 36 and optionally with the driving device 32. The communication between the controller 38, the rheological probe 36 and the driving device 32 can be provided by a wireless connection, a wired connection, or a combination thereof. In some embodiments, the controller 38 is configured to receive the pressure values, and associated circumferential positions and/or timestamps, from the rheological probe 36. The pressure values can be stored on a memory system of the controller 38 in some embodiments. The pressure values may also be communicated to an external device or network. In some embodiments, the torque applied on the drum 16 to drive its rotation can be controlled by the controller 38. Additionally or alternately, the torque applied on the drum 16 can be manually controlled by the driver using a hand lever.

In some embodiments, upon receiving an instruction to reduce the rotational speed of the drum 16 to the low rotation speed, e.g., via the hand lever, the system 30 can initiate the sequence for detecting and monitoring segregation occurring in the fresh concrete mixture 12 being agitated in the drum 16, as will be described below. Broadly described, the controller 38 is configured to receive the measured pressure values from the rheological probe, access reference data indicative of a behaviour of the rheological probe moving through a fresh concrete mixture of a non-segregated state, and to detect the segregation by comparing at least some of the measured pressure values to the reference data. In some embodiments, the controller 38 is configured to determine a degree of segregation of the fresh concrete mixture 12 based on the comparison, and generate an output corresponding to the determined degree of segregation. Upon the determined degree of segregation being above a predetermined threshold, the output can be indicative of an alert.

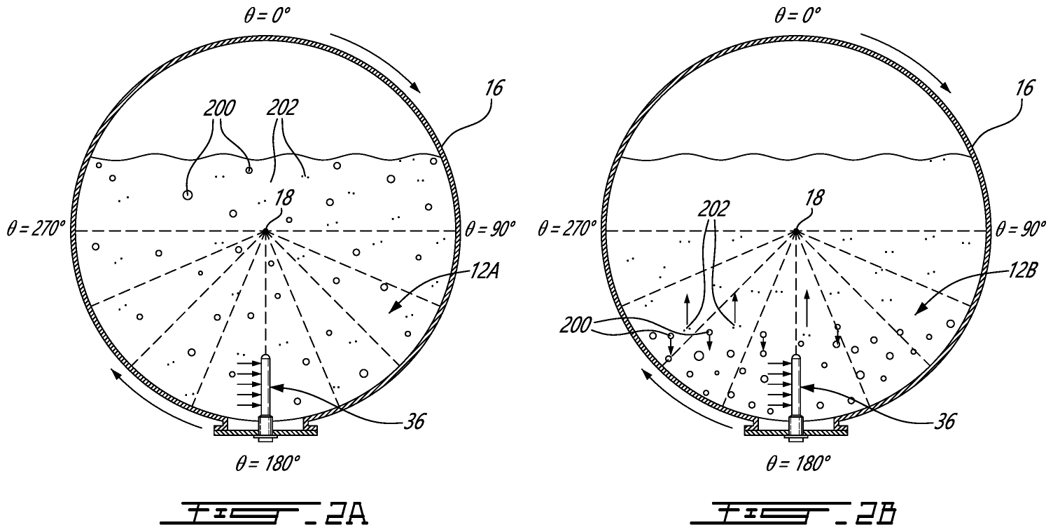

Reference is now made to FIGS. 2A and 2B which show sectional views taken along line 2-2 of FIG. 1. On one hand, FIG. 2A shows the drum 16 agitating a fresh concrete mixture 12A of a non-segregated state whereas FIG. 2B shows the drum 16 agitating a fresh concrete mixture 12B of a segregated state. It will be appreciated that when no segregation occurs, such as shown in FIG. 2A, the denser concrete ingredients 200 and the lighter concrete ingredients 202 are somewhat homogeneously suspended in the fresh concrete mixture 12A. However, in situations where segregation arises gravity pulls the denser concrete ingredients 200 down towards the bottom of the suspension, and reciprocally pushes the lighter concrete ingredients 202 upwards, as best shown in FIG. 2B. As the segregating fresh concrete mixture 12B may lead to poor hardened concrete performance, detecting segregation during the agitation phase is of importance.

To detect segregation occurring in a fresh concrete mixture as it is being agitated in the drum 16, the pressure values measured by the rheological probe 36 can advantageously be used to identify segregation indication(s) by comparing some of the measured pressure values to corresponding reference data including threshold(s), example of which are described below for convenience.

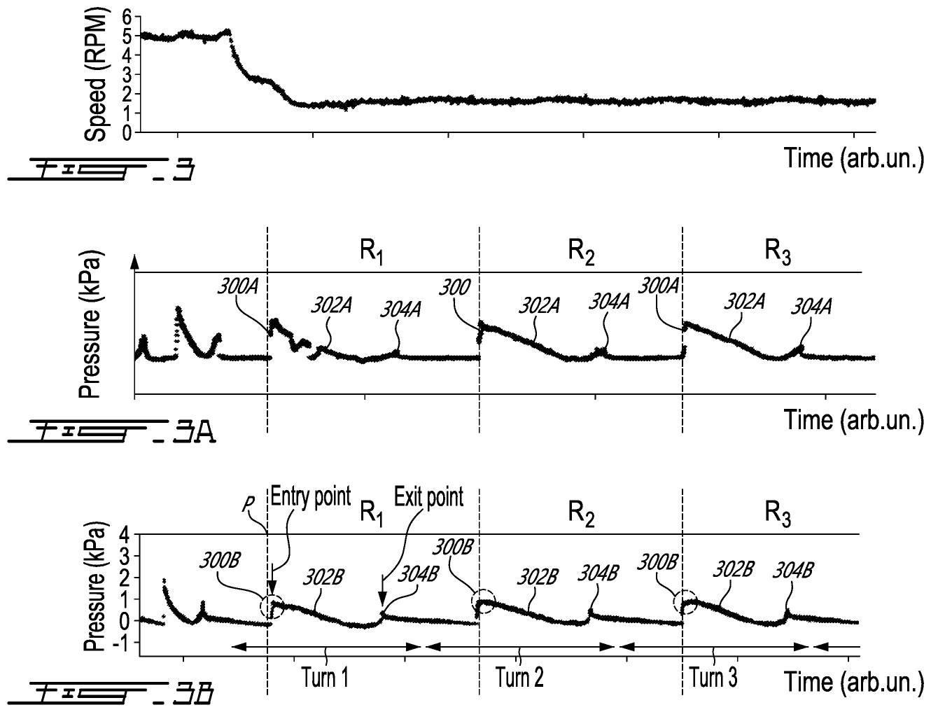

FIG. 3 shows a graph of the rotational speed of the drum as a function of time during pressure measurements for fresh concrete mixtures 12A and 12B of FIGS. 2A and 2B. The measured pressure values for each fresh concrete mixture are shown in FIGS. 3A and 3B, respectively.

As can be appreciated in FIG. 3A, at each rotation Ri of the drum during the agitation phase (with i denoting an integer), the pressure values have a steep increase 300A followed by a negative slope 302A until a trail decrease 304A occurs. As the steep increase 300A is indicative of the rheological probe entering in the fresh concrete mixture, the trail decrease is indicative of the rheological probe exiting the fresh concrete mixture. The pressure values in-between are indicative of the resistive pressure that the fresh concrete mixture offers. When the rheological probe exits the concrete, the measures pressure values are significantly lower as the rheological probe travels through air for a given period of time, until it enters the fresh concrete mixture again in a successive rotation of the drum, and so forth. It was found that some discrepancies in the measured pressure values can be observed depending on whether the rheological probe travels in a non-segregating fresh concrete mixture or in a segregating fresh concrete mixture, such as shown in FIGS. 3A and 3B. Based on these discrepancies, one can detect segregation occurring in the fresh concrete mixture while it is being agitated in the drum, and prior to the pouring of the fresh concrete mixture at a job site. For instance, although a steep increase 300B, a gradual decreasing slope 302B and a trail decrease 304B can also be observed in the pressure values of FIG. 3B, the discrepancies in the maximal pressure values of the steep increases 300A and 300B, the slope values of the negative slopes 302A and 302B, the maximal pressure values of the steep decreases 304A and 304B are quantifiable and comparable. As can be appreciated, the reference data can be indicative of a behaviour of the rheological probe in a fresh concrete mixture in absence of segregation.

In some embodiments, the reference data include one or more pressure values measured during the first rotation of the drum after the rotational speed has been reduced from the high rotational speed to the low rotational speed, i.e., during the first rotation of the drum at the low rotational speed. The reference data can also include pressure value(s) measured during one or more rotations of the drum immediately following the reducing of the rotational speed from the high rotational speed to the low rotational speed. As illustrated in FIG. 3B, the reference data can include the pressure value(s) measured during the rotation R1. In these embodiments, the pressure value(s) measured during the rotations R2, R2, Ri (with i>2) are compared to the pressure value(s) measured during the first rotation R1, as the concrete ingredients may not have started to segregate yet at that point in time.

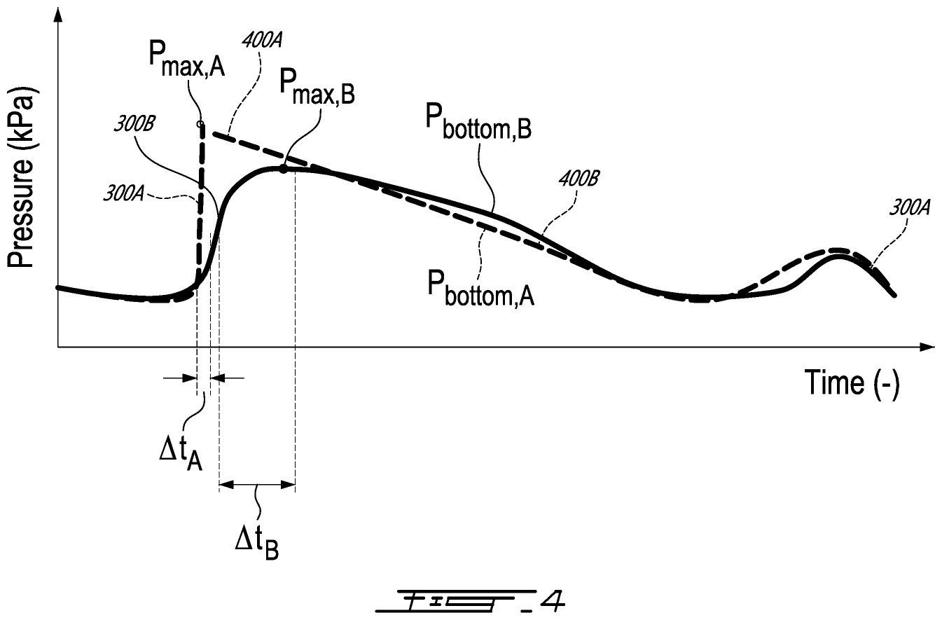

As shown in FIG. 4, these discrepancies are best appreciated by overlapping a first pressure value pattern 400A pertaining to a fresh concrete mixture of a non-segregated state to a second pressure value pattern 400B pertaining to a fresh concrete mixture of a segregated state. For instance, one can appreciate a significant difference in the maximal pressure values Pmax,A and Pmax,B associated to the steep increases 300A and 300B, with the maximal pressure value Pmax,A being significantly greater than the maximal pressure value Pmax,B. This difference may result from the rheological probe hitting, upon entry in the fresh concrete mixture, a greater amount of denser concrete ingredients in the case of the non-segregating fresh concrete mixture than it would in the case of the segregating fresh concrete mixture, as denser concrete ingredients are settled at the bottom of the drum, far from the entry point of the rheological probe. The first and second pressure patterns 400A and 400B also show a first shift ΔtA between the steep increase 300A and the maximal pressure value Pmax,A for the first pressure pattern 400A, and a second time shift ΔtB between the steep increase 300B and the maximal pressure Pmax,B for the second pressure pattern 400B. As shown, one may appreciated that the second time shift ΔtB is significantly greater than the first time shift ΔtA. This may be justified by the rheological probe moving through a greater amount of smaller and lighter ingredients immediately as it enters the segregating fresh concrete mixture, thereby amounting to a smoother pressure variation. Moreover, the pressure value Pbottom,B measured when the rheological probe is at the bottom of the drum appears to be greater in the second pressure pattern 400B, e.g., as the rheological probe is expected to move through and hit the denser concrete ingredients of the segregating fresh concrete mixture.

Referring back to FIG. 1, the controller 38 is configured identify segregation indication(s) based on the aforementioned quantifiable discrepancies, and more specifically by comparing some of the measured pressure values to corresponding reference data and corresponding threshold(s). The mixer truck 10 has a user interface 40 which is communicatively coupled with the controller 38. As can be understood, the user interface 40 can be used to display data and/or receive inputs. Examples of data that can be displayed by the user interface 40 can include an indication whether segregation has been detected, the instantaneous degree of segregation of the fresh concrete mixture, the torque at which the driving device 32 currently drives rotation of the drum 16, the instantaneous speed of rotation of the drum 16, and the like. Examples of inputs that can be received via the user interface 40 can include an instruction regarding the speed at which the drum 16 is to be rotated and the like. In some embodiments, the user interface 40 has a speed actuator 42 within a cabin 44 of the mixer truck 10. In these embodiments, the speed actuator 42 is actuatable to increase or decrease the rotational speed of the drum 16 by moving the speed actuator 42 up or down, as desired. For instance, the rotational speed can be increased upon detecting segregation occurring in the fresh concrete mixture.

The mixer truck 10 has at least a rotational speed sensor 46 to monitor the rotational speed of the drum 16. More specifically, the rotational speed sensor 46 measures a plurality of speed values Si indicative of the speeds at which the drum 16 rotates during the given period of time, and generates a signal based on the measured speed values. Any suitable rotational speed sensor can be used. In this example, the controller 38 is communicatively coupled with the rotational speed sensor 46. The communication between the controller 38 and the rotational speed sensor 46 can be provided by a wireless connection, a wired connection, or a combination thereof. In some embodiments, the detection of the segregation and the monitoring of the degree of segregation of the fresh concrete mixture are initiated only when the measured speed value is below a given threshold.

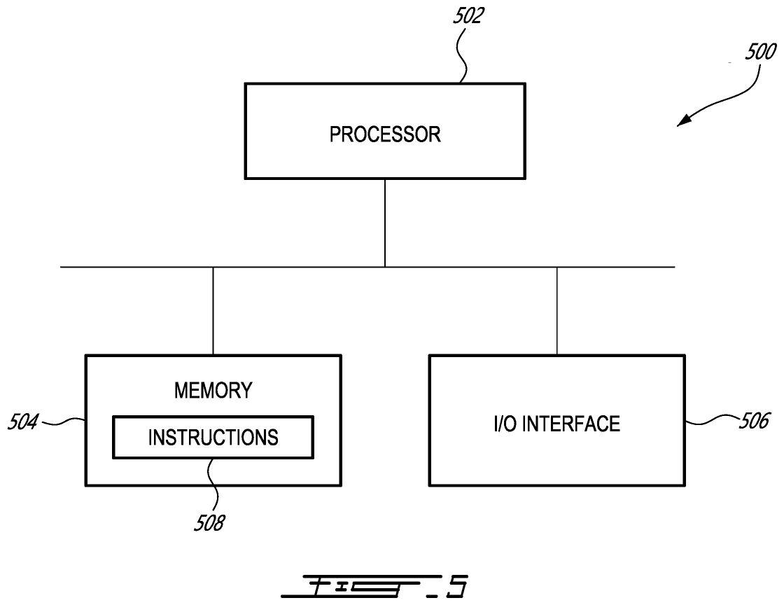

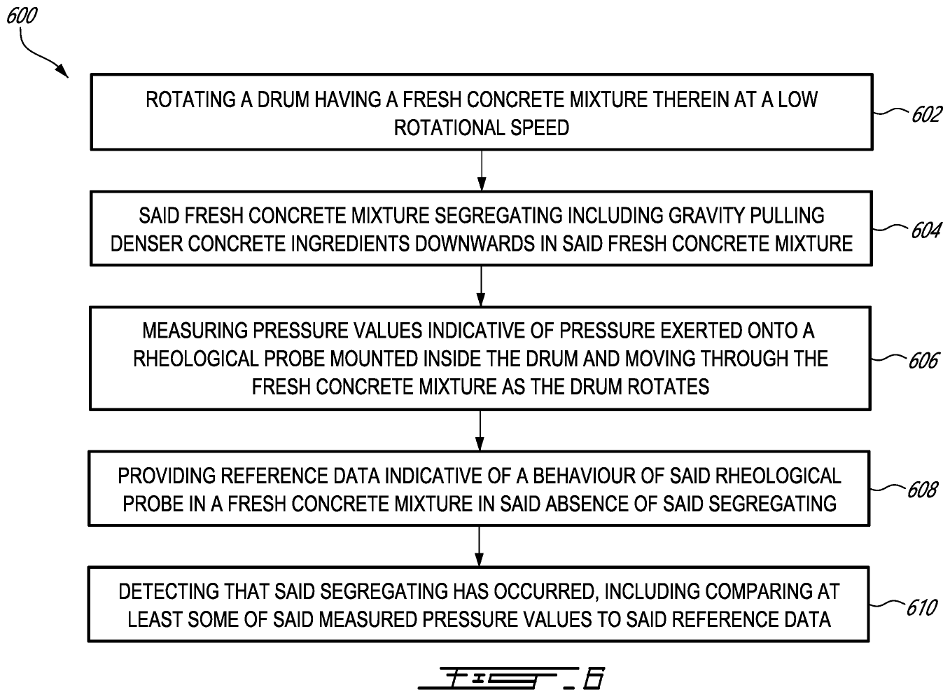

The controller 38 can be provided as a combination of hardware and software components. The hardware components can be implemented in the form of a computer device 500, an example of which is described with reference to FIG. 5. Moreover, the software components of the controller 38 can be implemented in the form of a software application performing executable method steps, example of which being described with reference to FIG. 6.

Referring to FIG. 5, the computer device 500 can have a processor 502, a memory 504, and I/O interface 506. Instructions 508 for determining the degree of segregation based on measured pressure values and corresponding threshold(s) can be stored on the memory 504 and accessible by the processor 502.

The processor 502 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 504 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 506 enables the computer device 500 to interconnect with one or more input devices, such as keyboard(s), mouse(s), rheological probe(s), rotational speed sensor(s), hydraulic pressure sensor(s), or with one or more output devices such as local or remote display(s), local or remote memory system(s), external network such as the Internet.

Each I/O interface 506 enables the controller 38 to communicate with other components, to exchange data with other components, to access and connect to network resources, to server applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The computer device 500 and any complementary software application described above are meant to be examples only. Other suitable embodiments of the controller 38 can also be provided, as it will be apparent to the skilled reader.

FIG. 6 shows a flow chart of an example of a method 600 for detecting segregation occurring in a fresh concrete mixture being agitated in a drum.

At step 602, the drum is rotated about the rotation axis at a low rotational speed for agitating said fresh concrete mixture during at least a rotation. Examples of such low rotation speeds can include, but not limited to, 1 RPM, 2, RPM, 3 RPM and up to 4 RPM. In some embodiments, the step of rotating the drum includes a step of reducing the rotational speed of the drum from a high rotational speed for mixing the concrete ingredients to one another to a low rotational speed for agitating the fresh concrete mixture. The speed reduction step can be performed upon determining that the mixing phase is satisfactorily completed and is maintained until the concrete mixer truck arrives at a job site. It was found that when the rotational speed of the drum is reduced suddenly, e.g., due to a driver intervention or a speed control device activation, the measure pressure values stabilize quickly unless the fresh concrete mixture is prone to segregation.

At step 604, the fresh concrete mixture segregates. At this step, denser concrete ingredients of the fresh concrete mixture are pulled downwards in the fresh concrete mixture thanks to the gravity. Reciprocally, lighter concrete ingredients may be pushed upwards in the fresh concrete mixture.

At step 606, pressure values are measured using a rheological probe mounted inside the drum. The measured pressure values are indicative of pressure exerted onto the rheological probe as it moves through the fresh concrete mixture upon rotation of the drum. In some embodiments, the pressure values are measured during the mixing phase, i.e., when the drum is rotated at the high rotational speed, as well as during the subsequent, agitation phase, i.e., when the drum is rotated at the low rotational speed. The pressure values are typically measured at a given frequency. For instance, the frequency can range between 1 Hz and 100 Hz, depending on the embodiment. The measured pressure values can be communicated to the controller, or stored on a memory thereof, in real time or quasi real time in some embodiments. In some embodiments, the pressure values may be communicated to the controller in batch. It was found that differences can be observed between non-segregating and segregating fresh concrete mixtures, e.g., the measured pressure value measured at the top of the fresh concrete mixture is progressively reduced while the pressure value measured at the bottom of the drum is progressively increased, as discussed above.

At step 608, reference data are provided. The reference data are indicative of a behaviour of the rheological probe in a fresh concrete mixture in the absence of segregation. For instance, the reference data can be indicative of a behaviour of the rheological probe in the fresh concrete mixture during a rotation of the drum immediately following the reducing of the rotational speed of the drum from the high rotational speed to the low rotational speed. The reference data can include reference pressure values indicative of pressure exerted onto a reference rheological probe mounted inside a reference drum and moving through fresh concrete mixture of a non-segregated state during one or more drum rotation. The reference data may be accessed by the controller or otherwise retrieved from an accessible memory. In some embodiments, there may be a number of reference data sets to chose from, with each reference data set comprising reference pressure values measured using a reference rheological probe under different parameters including, but not limited to, rotational speed, mixture composition information, drum characteristics and/or volume. In these embodiments, the step of accessing may include a step of selecting the reference data among the different reference data sets based on matching parameters such as matching low rotational speeds, matching composition data, matching drum characteristics and matching fresh concrete mixture volume.

The reference data can include one or more different types of thresholds. In some embodiments, the threshold is a maximal pressure value threshold. In these embodiments, the step of comparing includes a step of comparing a maximal one of the measured pressure values to the maximal pressure value threshold. Segregation may be detected when the maximal pressure value is below the maximal pressure value threshold. In some embodiments, the threshold is a bottom pressure value threshold. In these embodiments, the step of comparing includes a step of identifying a bottom pressure value associated to a measurement made when the rheological probe is at or about a bottom of the drum, and comparing the bottom pressure value to the bottom pressure value threshold. In this case, segregation may be detected when the bottom pressure value is greater than the bottom pressure value threshold. In some embodiments, the threshold is a slope threshold. In these embodiments, the step of comparing includes a step of finding a set of the measured pressure values associated to the rheological probe entering the fresh concrete mixture and moving through fresh concrete mixture for a given period of time, and comparing a slope value of these pressure values of the set to the slope threshold. In this case, segregation may be detected when the slope value is below the slope threshold. In some embodiments, the threshold is a time threshold. In these embodiments, the step of comparing includes a step of finding a time stamp difference between a time stamp associated with a middle of the set of measured pressure values associated to the rheological probe entering the fresh concrete and a timestamp of the maximal pressure value. Instead of timestamps, measurements index numbers may also be used. In these embodiments, segregation may be detected when the time stamp difference is greater than a time threshold. Additionally or alternately, the whole pressure pattern formed by the measured pressure values can be compared to reference pressure patterns to detect segregation. In some embodiments, these patterns are characterized by mathematical parameters such as area under the curve for one or more sectors or by other parameters that can suitably describe the shape of the patterns. In some embodiments, the controller includes a segregation detection module being trained using machine learning algorithms and adapted to recognize segregation indication(s) in the measured pressure value(s). In these embodiments, the reference data can include training data on which the segregation detection module relies in the segregation detection.

At step 610, segregation occurring in the fresh concrete mixture, if any, is detected by comparing at least some of the measured pressure values to the reference data and/or associated threshold(s). In some embodiments, the step of comparing includes comparing at least some pressure values measured in a subsequent rotation of the drum to at least some pressure values measured in a previous rotation of the drum. Typically, the previous rotation of the drum can be a rotation where segregation is expected to not have occurred yet, e.g., during the first rotation (and/or second rotation) of the drum immediately following the reducing of the rotational speed of the drum. In some embodiments, a degree of segregation is determined. In some embodiments, the degree of segregation is a binary degree, with a first binary degree (e.g., 0) indicating that the fresh concrete mixture is non-segregating and a second binary degree (e.g., 1) indicating that the fresh concrete mixture is segregating. In some other embodiments, the degree of segregation can be a value ranging on a given segregation scale. For instance, 0 may indicate that the fresh concrete mixture is non-segregating and 10 may indicate that the fresh concrete mixture is segregating. The scale can depend on a quantification of how much the measured pressure value(s) or slope(s) differ from the corresponding threshold(s).

In some embodiments, the method 600 can include a step of generating an output indicative of whether segregation has been detected, and/or of the degree of segregation. In some embodiments, the output is provided in the form of an alarm to be display in the cabin of the concrete mixer truck. In some embodiments, the output is an alarm which is stored on a memory of the controller for later consultation. In some embodiments, once such an alarm is generated, the concrete mixer truck may be instructed to go back to the plant instead of pouring the segregating fresh concrete mixture. In some embodiments, for instance if the fresh concrete mixture is segregating because it has too much entrained-air, an admixture reducing the air content can be added to the fresh concrete mixture. In some embodiments, for instance if the fresh concrete mixture is segregating because it has too plasticising admixture, additional cement (or sand) can be added to the fresh concrete mixture. In some embodiments, it is determined that the fresh concrete mixture is segregating because it has too much water, in these cases the segregating fresh concrete mixture can be returned to the plant. In some embodiments, the output, if generated prior to the concrete mixer truck leaving the concrete plant, could be indicative of correction measure(s) to be performed on the fresh concrete. Such correction measure(s) can include, but are not limited to, adding concrete ingredients (e.g., dry material) inside the drum, mixing the drum for a longer period of time and the like.

As can be understood, the examples described above and illustrated are intended to be exemplary only. Although the examples described above are shown in the context of a concrete mixer truck, the drum can also pertain to a stationary mixer and the like. Accordingly, the methods and systems described herein are not to be limited to concrete mixer trucks. The scope is indicated by the appended claims.

What is claimed is:

1. A method for detecting segregation occurring in a fresh concrete mixture being agitated in a drum, said drum having a rotation axis, said fresh concrete mixture having denser concrete ingredients, said method comprising:
rotating said drum about said rotation axis at a given rotational speed for agitating said fresh concrete mixture during at least a rotation;
said fresh concrete mixture segregating, said segregating including gravity pulling said denser concrete ingredients downwards in said fresh concrete mixture;
measuring a plurality of pressure values indicative of pressure exerted onto a rheological probe mounted inside said drum and moving through said fresh concrete mixture as said drum rotates;
providing reference data indicative of a behaviour of said rheological probe in a fresh concrete mixture in said absence of said segregating; and
detecting that said segregating has occurred, including comparing at least some of said measured pressure values to said reference data.

2. The method of claim 1 wherein, prior to said segregating, said fresh concrete mixture is in a non-segregated state in which said rheological probe has a behaviour indicative of said reference data.

3. The method of claim 1 wherein, prior to said rotating, said rotating including reducing a rotational speed of said drum from a high rotational speed to said given rotational speed, said segregating occurring during said rotating at said given rotational speed.

4. The method of claim 3 wherein said reference data are indicative of a behaviour of said rheological probe in the fresh concrete mixture during one or more rotations of said drum immediately following said reducing.

5. The method of claim 1 wherein upon detecting that said segregating has occurred, generating an alert indicative that said fresh concrete mixture is in a segregated state.

6. The method of claim 1 wherein said reference data includes a maximal pressure value threshold, said comparing including comparing a maximal one of said measured pressure values to said maximal pressure value threshold.

7. The method of claim 1 wherein said reference data includes a bottom pressure value threshold, said comparing including comparing one of said pressure values associated to a pressure value measured as said rheological probe is at a bottom of said drum to said bottom pressure value threshold.

8. The method of claim 1 wherein said reference data includes a slope threshold, said comparing including determining a slope of a set of said pressure values associated with said rheological probe entering said fresh concrete mixture, and comparing said slope value to said slope threshold.

9. The method of claim 1 wherein said reference data includes reference pressure values indicative of pressure exerted onto a reference rheological probe mounted inside a reference drum and moving through a fresh concrete mixture of a non-segregated state as said reference drum rotates.

10. The method of claim 1 wherein said providing includes selecting said reference data among a plurality of different sets of reference data based on at least one of matching low rotational speeds, matching composition data, matching drum characteristics and matching fresh concrete mixture volume.

11. The method of claim 1 wherein said detecting includes determining a degree of segregation of said fresh concrete mixture.

12. The method of claim 11 wherein said degree of segregation is a value ranging on a given scale.

13. The method of claim 11 wherein said reference data are indicative of a behaviour of said rheological probe in the fresh concrete mixture in a previous rotation of said drum, said comparing including comparing at least some pressure values measured in a subsequent rotation of said drum to at least some pressure values measured in said previous rotation of said drum.

14. A system for detecting segregation occurring in a fresh concrete mixture being agitated in a drum, said drum having a rotation axis, said fresh concrete mixture having denser concrete ingredients, said system comprising:
a driving device driving rotation of said drum about said rotation axis at a low rotational speed for agitating said fresh concrete mixture during at least a rotation, said fresh concrete mixture segregating including gravity pulling said denser concrete ingredients downwards in said fresh concrete mixture;
a rheological probe mounted inside said drum and moving through said fresh concrete mixture as said drum rotates, and measuring a plurality of pressure values indicative of pressure exerted onto said rheological probe during said rotation; and
a controller being communicatively coupled to said rheological probe, said controller having a processor and a memory having stored thereon instructions that when executed by said processor perform the steps of:
accessing reference data indicative of a behaviour of said rheological probe in a fresh concrete mixture in said absence of said segregating; and
detecting that said segregating has occurred, including comparing at least some of said measured pressure values to said reference data.

15. The system of claim 14 wherein, prior to said segregating, said fresh concrete mixture is in a non-segregated state in which said rheological probe has a behaviour indicative of said reference data.

16. The system of claim 14 wherein, prior to said rotating, said rotating including reducing a rotational speed of said drum from a high rotational speed to said low rotational speed, said reducing causing said segregating.

17. The system of claim 16 wherein said reference data are indicative of a behaviour of said rheological probe in the fresh concrete mixture during one or more rotations of said drum immediately following said reducing.

18. The system of claim 14 wherein upon detecting that said segregating has occurred, generating an alert indicative that said fresh concrete mixture is in a segregated state.

19. The system of claim 14 wherein said reference data includes a maximal pressure value threshold, said comparing including comparing a maximal one of said measured pressure values to said maximal pressure value threshold.

20. The system of claim 14 wherein said reference data includes a bottom pressure value threshold, said comparing including comparing one of said pressure values associated to a pressure value measured as said rheological probe is at a bottom of said drum to said bottom pressure value threshold.

21. The system of claim 14 wherein said reference data includes reference pressure values indicative of pressure exerted onto a reference rheological probe mounted inside a reference drum and moving through a fresh concrete mixture of a non-segregated state as said reference drum rotates.

22. The system of claim 14 further comprising a display displaying whether said segregating has occurred.

23. The system of claim 14 wherein said controller is further configured to generate an alert indicative of whether said segregating has occurred.

24. The system of claim 14 wherein said reference data are indicative of a behaviour of said rheological probe in the fresh concrete mixture in a previous rotation of said drum, said comparing including comparing at least some pressure values measured in a subsequent rotation of said drum to at least some pressure values measured in said previous rotation of said drum.
